(19) United States
(12) Patent Application Publication
(10) Pub. No.: US 2013/0197344 A1
(43) Pub. Date: Aug. 1, 2013

(54) PHOTO-ACOUSTIC TOMOGRAPHY

(71) Applicant: Electronics and Telecommunications Research Institute, Daejeon (KR)

(72) Inventors: Han Young YU, Daejeon (KR); Yark Yeon KIM, Daejeon (KR); Yong Ju YUN, Daejeon (KR); Won Ick JANG, Daejeon (KR)

(73) Assignee: Electronics and Telecommunications Research Institute, Daejeon (KR)

(21) Appl. No.: 13/748,537

(22) Filed: Jan. 23, 2013

(30) Foreign Application Priority Data
Jan. 30, 2012 (KR) 10-2012-0009085
May 30, 2012 (KR) 10-2012-0057224

(51) Int. Cl. A61B 5/00

(52) U.S. Cl. CPC A61B 5/0095
USPC 600/407

(57) ABSTRACT

The present invention relates to a photo-acoustic tomography that can acquire a functional image for an inner part of a living body through generation of a local ultrasonic wave generated by energy that is introduced from a laser light source, and to a photo-acoustic tomography using a semiconductor laser and an optical fiber power amplifying device in order to increase resolution and an image acquisition time of an image, a photo-acoustic tomography that can acquire a high-sensitive image even in a place where a penetration depth is large through energy modulation, and a high-sensitive high-speed photo-acoustic tomography that can acquire a high-speed image by placing an array-type laser light source.

### CROSS-REFERENCE TO RELATED APPLICATIONS

This application claims priority to and the benefit of Korean Patent Application No. 10-2012-0009085 filed in the Korean Intellectual Property Office on Jan. 30, 2012 and Korean Patent Application No. 10-2012-0057224 filed in the Korean Intellectual Property Office on May 30, 2012, the entire contents of which are incorporated herein by reference.

### TECHNICAL FIELD

The present invention relates to a photo-acoustic tomography that can acquire a functional image for an inner part of a living body through generation of a local ultrasonic wave generated by energy that is introduced from a laser light source, and to a photo-acoustic tomography using a semiconductor laser and an optical fiber power amplifying device in order to increase resolution and an image acquisition time of an image, a photo-acoustic tomography that can acquire a high-sensitive image even in a place where a penetration depth is large through energy modulation, and a high-sensitive high-speed photo-acoustic tomography that can acquire a high-speed image by placing an array-type laser light source.

### BACKGROUND ART

In the photo-acoustic tomography, when a light source having high energy, such as a laser is irradiated to a biomaterial during a pulse cycle and light energy is absorbed by the biomaterial to emit energy, an ultrasonic wave generated by rolling of cell lattices is propagated to the outside of the biomaterial and a functional structure of the biomaterial may be analyzed by measuring the ultrasonic wave.

Since absorbency of laser energy is different depending on a material type of the biomaterial, the ultrasonic wave is frequently jetted as a material frequently absorbs the laser energy at the time of analyzing the functional structure of the biomaterial by using the photo-acoustic tomography.

A part which can be measured most minutely in a photo-acoustic tomography which has been used in recent years is a blood vessel where the most hemoglobin is distributed. That is, a part where an absorption rate of a Nd:YAG laser which has been primarily used in recent years is highest is hemoglobin which is distributed a lot in the blood vessel. Since the distribution of the blood vessel is most formed when a tumor including a cancer is generated, the photo-acoustic tomography has the largest advantage to visualize a functional image of the tumor in real time therefrom.

In the related art, a laser that can best irradiate light energy is the Nd:YAG laser. However, it has been difficult to scan an inner part through a high speed because a switching speed is low as 10 to 15 Hz as well as a solid laser has a large volume.

The existing photo-acoustic tomography has had a disadvantage in which an image of a cell positioned at a deep position cannot be acquired as a depth in which the laser light can penetrate is maximally lowest as 5 to 7 cm in a bio cell. Since the amount of a generated ultrasonic wave is minute even though the laser light penetrates at the maximum depth, it is not easy to distinguish the ultrasonic wave from noise.

In the existing photo-acoustic tomography, since it takes a lot of time to acquire the image of the bio cell, there is a problem that there are a lot of limitations in utilizing the existing photo-acoustic tomography.

### SUMMARY OF THE INVENTION

The present invention has been made in an effort to provide a photo-acoustic tomography that increases a scan speed by increasing a switching speed while minimizing a system, minimizes a problem caused due to noise by maximizing a penetration depth of a light source, and minimizes a time required to acquire an image.

An exemplary embodiment of the present invention provides a photo-acoustic tomography, including: a light source outputting light; an amplification unit amplifying and outputting the light output from the light source to be absorbed in a biomaterial which is an inspection target; a sensing unit sensing an ultrasonic wave generated as the light output from the amplification unit is absorbed in the biomaterial; and an image implementing unit implementing an image of an inner part of the biomaterial by using the ultrasonic wave sensed by the sensing unit.

Another exemplary embodiment of the present invention provides a photo-acoustic tomography, including: a first light source outputting first light to be absorbed in a biomaterial which is an inspection target; a second light source outputting second light which has power equal to or lower than the power of the first light and has a lower frequency than the first light to be absorbed in the biomaterial; a modulation unit controlling the power and the frequency of the second light output from the second light source; a sensing unit sensing an ultrasonic wave generated as the first light and the second light are absorbed in the biomaterial; and an image implementing unit implementing an image of an inner part of the biomaterial by using the ultrasonic wave sensed by the sensing unit.

Yet another exemplary embodiment of the present invention provides a photo-acoustic tomography, including: a light source array including first to n-th light sources outputting first to n-th light to be absorbed in the biomaterial which is an inspection target; a sensor array including first to m-th sensors sensing ultrasonic waves generated as the first to n-th light is absorbed in the biomaterial; and an image implementing unit implementing an image of an inner part of the biomaterial by using the ultrasonic waves sensed by the sensor array.

A photo-acoustic tomography according to the exemplary embodiment of the present invention having the above configuration can provide the following effects.

First, the photo-acoustic tomography according to the present invention can configure a system having a high scan speed and high mobility by using a minimized light source and a light source having a high switching speed.

Second, the photo-acoustic tomography according to the present invention can acquire an ultrasonic signal having high sensitivity and a low-noise signal by modulating the light source.

Third, the photo-acoustic tomography according to the present invention can acquire a high-speed image by scanning a light source in a living body at a high speed through a light source which is formed in an array pattern.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

FIG. 1 is a block diagram illustrating a photo-acoustic tomography according to a first exemplary embodiment of the present invention.

FIG. 2 is a block diagram illustrating a photo-acoustic tomography according to a second exemplary embodiment of the present invention.

FIG. 3 is a conceptual diagram for describing the photo-acoustic tomography of FIG. 2.

FIG. 4 is a block diagram illustrating a photo-acoustic tomography according to a third exemplary embodiment of the present invention.

FIG. 5 is a conceptual diagram for describing the photo-acoustic tomography of FIG. 4.

It should be understood that the appended drawings are not necessarily to scale, presenting a somewhat simplified representation of various features illustrative of the basic principles of the invention. The specific design features of the present invention as disclosed herein, including, for example, specific dimensions, orientations, locations, and shapes will be determined in part by the particular intended application and use environment.

In the figures, reference numbers refer to the same or equivalent parts of the present invention throughout the several figures of the drawing.

### DETAILED DESCRIPTION

A photo-acoustic tomography according to exemplary embodiments of the present invention will be described with reference to the accompanying drawings.

When it is determined that the detailed description of the known art related to the present invention may obscure the gist of the present invention, the detailed description thereof will be omitted.

In the exemplary embodiments, components and features of the present invention are combined with each other in a predetermined pattern. Each component or feature may be considered to be selective as long as not particularly explicitly mentioned. Each component or feature may be implemented in such a way that the corresponding component or feature is not combined with another component or feature. Some components and/or features are combined with each other to configure the exemplary embodiments of the present invention. The order of operations described in the exemplary embodiments of the present invention may be changed. Some component or feature of a predetermined exemplary embodiment may be included in another exemplary embodiment or may be replaced by a corresponding component or feature of another exemplary embodiment.

The exemplary embodiments of the present invention may be implemented through various means. For example, the exemplary embodiments may be implemented through hardware, firmware, software, or combinations thereof.

In the case of implementation of hardware, a method according to the exemplary embodiments of the present invention may be implemented by one or more ASICs (application specific integrated circuits), DSPs (digital signal processors), DSPDs (digital signal processing devices), PLDs (programmable logic devices), FPGAs (field programmable gate arrays), processors, controllers, microcontrollers, microprocessors, and the like.

In the case of implementation of the firmware or software, the method according to the exemplary embodiments of the present invention may be implemented by modules, procedures, or functions that perform the aforementioned functions or operations. A software code is stored in a memory unit to be driven by a processor. The memory unit is positioned in or outside the processor to transmit and receive data to and from the processor by various means which have already been known.

Specific terms used in the following description are provided to help understanding the present invention and the use of the specific terms may be changed to another pattern within a scope without departing from the spirit of the present invention.

### First Exemplary Embodiment

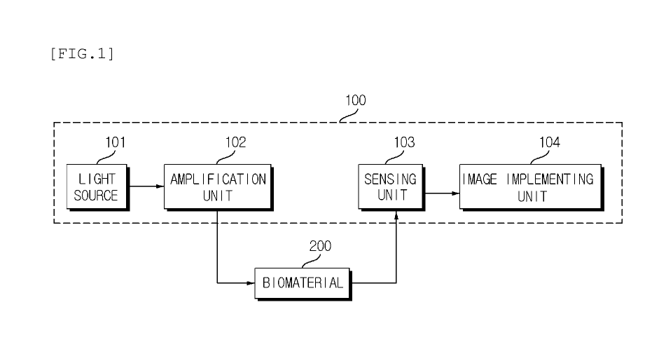

Hereinafter, a photo-acoustic tomography according to a first exemplary embodiment of the present invention will be described in detail with reference to FIG. 1.

FIG. 1 is a block diagram illustrating a photo-acoustic tomography 100 according to a first exemplary embodiment of the present invention.

Referring to FIG. 1, the photo-acoustic tomography 100 includes a light source 101 outputting light; an amplification unit 102 amplifying and outputting the light output from the light source 101 to be absorbed in a biomaterial 200 which is an inspection target; a sensing unit 103 sensing an ultrasonic wave generated as the light output from the amplification unit 102 is absorbed in the biomaterial; and an image implementing unit 104 implementing an image of an inner part of the biomaterial 200 by using the ultrasonic wave sensed by the sensing unit 103.

Various examples of the light source 101 are available within a scope without departing from the spirit of the present invention, but hereinafter, a case in which the light source 101 is a semiconductor laser will be described as an example in describing the photo-acoustic tomography 100 according to the first exemplary embodiment of the present invention.

In the photo-acoustic tomography 100 according to the first exemplary embodiment of the present invention, since the semiconductor laser adopted as the light source 101 has a high switching speed of 1 kHz or more, a low switching speed of a Nd:YAG laser which has been primarily used before can be overcome.

Since the intensity of light output from the semiconductor laser does not have high power enough to generate an ultrasonic wave as the light is irradiated to a biomaterial (that is, a cell), the photo-acoustic tomography 100 according to the first exemplary embodiment of the present invention includes an amplifier 102 as means for increasing the low power. Since the switching speed is kept to 1 kHz or more even though the amplifier 102 is provided, a problem caused due to a decrease in switching speed does not occur.

The amplifier 102 may be an optical-fiber optical amplifier for amplifying the light output from the light source 101. In the photo-acoustic tomography 100 according to the first exemplary embodiment of the present invention, the case in which the amplifier 102 is the optical-fiber optical amplifier has been described as an example, but the present invention is not limited thereto and various examples of the amplifier 102 are available within a scope without departing from the spirit of the present invention.

When the light emitted from the light source 101 and amplified through the amplifier 102 is absorbed in the biomaterial 200 to emit energy, a cell lattice swings to generate an ultrasonic wave and the ultrasonic wave is sensed by the sensing unit 103.

Information on the ultrasonic wave sensed by the sensing unit 103 is provided to the image implementing unit 103 and the image implementing unit 104 may acquire a functional image of the biomaterial 200 by configuring a 3D image by using the input ultrasonic wave information.

### Second Exemplary Embodiment

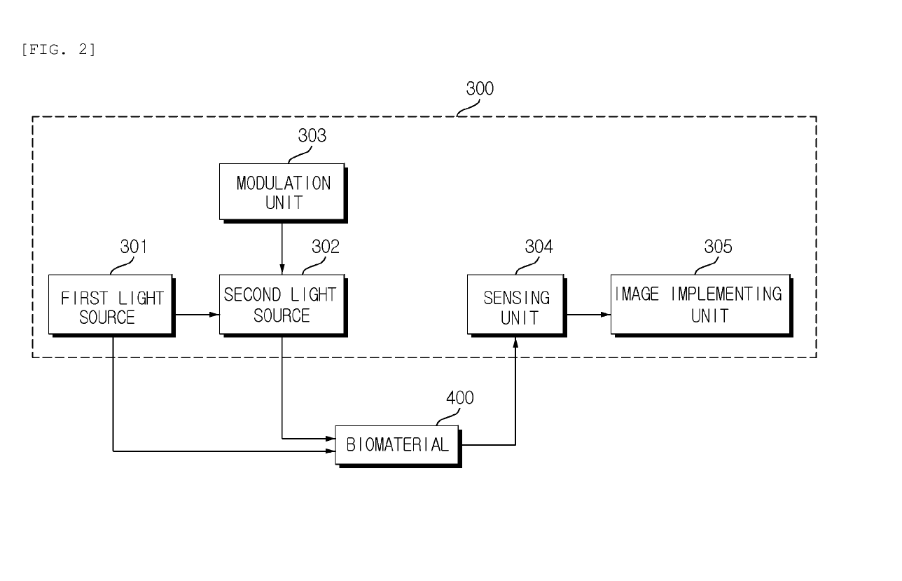

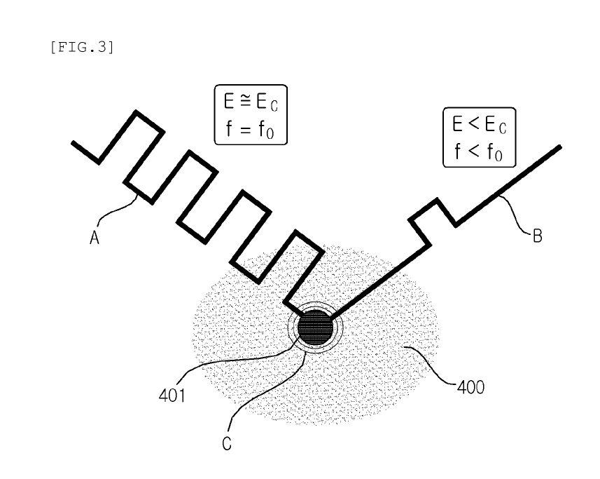

Hereinafter, a photo-acoustic tomography 300 according to a second exemplary embodiment of the present invention will be described in detail with reference to FIGS. 2 and 3.

FIG. 2 is a block diagram of the photo-acoustic tomography 300 according to the second exemplary embodiment of the present invention and FIG. 3 is a conceptual diagram for describing the photo-acoustic tomography 300 according to the second exemplary embodiment of the present invention.

The photo-acoustic tomography 300 according to the second exemplary embodiment of the present invention includes a light source 301 outputting first light (A of FIG. 3) to be absorbed in a biomaterial 400 which is an inspection target; a second light source 302 outputting second light (B of FIG. 3) which has power equal to or lower than the power of the first light and has a lower frequency than the first light to be absorbed in the biomaterial 400; a modulation unit 303 controlling the power and the frequency of the second light output from the second light source 302; a sensing unit 304 sensing an ultrasonic wave generated as the first light and the second light are absorbed in the biomaterial 400; and an image implementing unit 305 implementing an image of an inner part of the biomaterial 400 by using the ultrasonic wave sensed by the sensing unit 304.

Various examples of the first light source 301 and the second light source 302 are available within a scope without departing from the spirit of the present invention, but hereinafter, a case in which the first light source 301 and the second light source 302 are semiconductor lasers having a pulse type waveform will be described as an example in describing the present invention.

In the photo-acoustic tomography 300 according to the second exemplary embodiment of the present invention, when only the first light source 301 which is the semiconductor laser is provided, the light output from the first light source 301 has a limit in penetration depth and thus energy of the light is decreased when the light penetrates the biomaterial 400, and as a result, an irradiation method having high energy needs to be added. Therefore, to this end, the second light source 302 and the modulation unit 303 are additionally provided.

The second light source 302 may have power lower than the light output from the first light source 301 or equal to the light output from the first light source 301. As a result, the second light source does not generate the ultrasonic wave during propagation, and generates the ultrasonic wave only when the second light source 302 meets the light output from the first light source 301, and as a result, energy of both light overlaps.

The second light source 302 may have a frequency lower than the light output from the first light source 301. As a result, the light output from the second light source 302 is prevented from overlapping with the ultrasonic wave generated as the light output from the first light source 301 is absorbed in a biocell. That is, the generation of the ultrasonic wave by the light output from the first light source 301 is determined by the frequency of the light output from the first light source 301 and the generation of the ultrasonic wave is converted into the frequency of the light output from the first light source 301 to filter only the corresponding ultrasonic wave, but when the ultrasonic wave generated by the light output from the first light source 301 overlaps with the light output from the second light source 302, since the ultrasonic wave generated by the light output from the first light source 301 may not be distinguished from the ultrasonic wave generated by the light output from the second light source 302, the frequency of the light output from the second light source 302 is set to be lower than the frequency of the light output from the first light source 301, and as a result, both the ultrasonic waves may be distinguished from each other.

The ultrasonic wave generated by the light output from the first light source 301 and the ultrasonic wave generated by the light output from the second light source 302 are sensed by the sensing unit 304 and information on the ultrasonic waves sensed by the sensing unit 304 is provided to the image implementing unit 305. The image implementing unit 305 may acquire a functional image of the biomaterial 400 by configuring a 3D image by using the input ultrasonic wave information.

Although not illustrated in FIG. 2, the photo-acoustic tomography 300 according to the second exemplary embodiment of the present invention may further include an amplification unit (not illustrated) amplifying and outputting the first light output from the first light source 301.

### Third Exemplary Embodiment

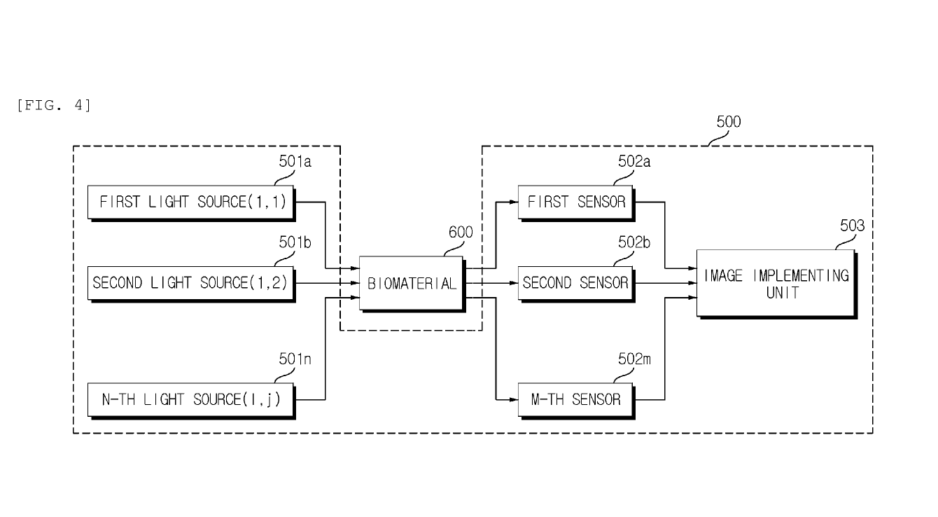

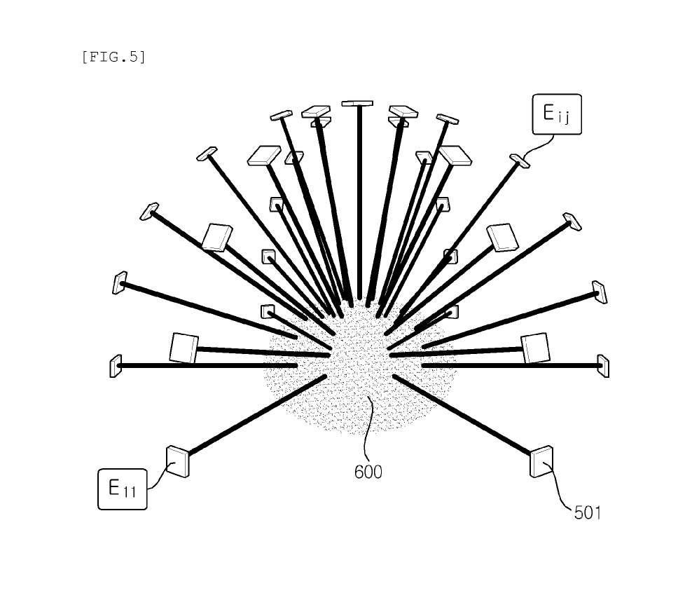

Hereinafter, a photo-acoustic tomography 500 according to a third exemplary embodiment of the present invention will be described in detail with reference to FIGS. 4 and 5.

FIG. 4 is a block diagram of the photo-acoustic tomography 500 according to the third exemplary embodiment of the present invention and FIG. 5 is a conceptual diagram for describing the photo-acoustic tomography 500 according to the third exemplary embodiment of the present invention.

The photo-acoustic tomography 500 according to the third exemplary embodiment of the present invention includes a light source array including first to n-th light sources 501a to 501n outputting first to n-th light to be absorbed in the biomaterial which is an inspection target; a sensor array including first to m-th sensors 502a to 502m sensing ultrasonic waves generated as the first to n-th light is absorbed in the biomaterial; and an image implementing unit 503 implementing an image of an inner part of the biomaterial 600 by using the ultrasonic waves sensed by the sensor array. Herein, n and m may be constants of 2 or more.

Various examples of the first to n-th light sources 501a to 501n are available within a scope without departing from the spirit of the present invention, but hereinafter, a case in which the first to n-th light sources 501a to 501n are the semiconductor lasers will be described as an example in describing the present invention.

In the photo-acoustic tomography 500 according to the third exemplary embodiment of the present invention, the first to n-th light sources 501a to 501n form an array pattern and are disposed to configure a part or the entirety of a sphere shape around the biomaterial 600 which is the inspection target.

The first to n-th light sources 501a to 501n are configured in the array pattern and a matrix pattern and may be disposed according to coordinates of (1,1), (1,2), , (i,j), respectively.

The first to n-th light sources 501a to 501n sequentially output light according to a predetermined order or an arbitrary order, respectively, and as a result, an effect as if multiple light sources are irradiated at one time may be obtained.

The first to n-th light sources 501a to 501n may have different power, and as a result, an image having high resolution may be implemented even with respect to a biomaterial having a long penetration depth.

The light source array constituted by the first to n-th light sources 501a to 501n may scan light to the biomaterial which is the inspection target at high speed, and as a result, when light is absorbed in the biomaterial, the ultrasonic wave is generated and the generated ultrasonic wave is sensed by the first to m-th sensors.

The first to m-th sensors 502a to 502m may be disposed in spaces among the first to n-th light sources 501a to 501n or integrally configured with the first to n-th light sources and this configuration has an advantage in simplification of the configuration and an advantage that a high-sensitivity ultrasonic image may be implemented as the sensors are adjacent to the light sources.

Information on the ultrasonic waves sensed by the first to m-th sensors 502a to 502m are provided to the image implementing unit 503 and the image implementing unit 503 configures a 3D image by using the input ultrasonic wave information to acquire a functional image of the biomaterial 600.

Although not illustrated in FIG. 4, the photo-acoustic tomography 500 according to the third exemplary embodiment of the present invention may further include an amplification unit (not illustrated) amplifying and outputting the light output from the first to n-th light sources 501a to 501n.

Meanwhile, the embodiments according to the present invention may be implemented in the form of program instructions that can be executed by computers, and may be recorded in computer readable media. The computer readable media may include program instructions, a data file, a data structure, or a combination thereof. By way of example, and not limitation, computer readable media may comprise computer storage media and communication media. Computer storage media includes both volatile and nonvolatile, removable and non-removable media implemented in any method or technology for storage of information such as computer readable instructions, data structures, program modules or other data. Computer storage media includes, but is not limited to, RAM, ROM, EEPROM, flash memory or other memory technology, CD-ROM, digital versatile disks (DVD) or other optical disk storage, magnetic cassettes, magnetic tape, magnetic disk storage or other magnetic storage devices, or any other medium which can be used to store the desired information and which can accessed by computer.

Communication media typically embodies computer readable instructions, data structures, program modules or other data in a modulated data signal such as a carrier wave or other transport mechanism and includes any information delivery media. The term “modulated data signal” means a signal that has one or more of its characteristics set or changed in such a manner as to encode information in the signal. By way of example, and not limitation, communication media includes wired media such as a wired network or direct-wired connection, and wireless media such as acoustic, RF, infrared and other wireless media.

Combinations of any of the above should also be included within the scope of computer readable media.

As described above, the exemplary embodiments have been described and illustrated in the drawings and the specification. The exemplary embodiments were chosen and described in order to explain certain principles of the invention and their practical application, to thereby enable others skilled in the art to make and utilize various exemplary embodiments of the present invention, as well as various alternatives and modifications thereof. As is evident from the foregoing description, certain aspects of the present invention are not limited by the particular details of the examples illustrated herein, and it is therefore contemplated that other modifications and applications, or equivalents thereof, will occur to those skilled in the art. Many changes, modifications, variations and other uses and applications of the present construction will, however, become apparent to those skilled in the art after considering the specification and the accompanying drawings. All such changes, modifications, variations and other uses and applications which do not depart from the spirit and scope of the invention are deemed to be covered by the invention which is limited only by the claims which follow.

What is claimed is:

1. A photo-acoustic tomography, comprising:
a light source outputting light;
an amplification unit amplifying and outputting the light output from the light source to be absorbed in a biomaterial which is an inspection target;
a sensing unit sensing an ultrasonic wave generated as the light output from the amplification unit is absorbed in the biomaterial; and
an image implementing unit implementing an image of an inner part of the biomaterial by using the ultrasonic wave sensed by the sensing unit.

2. The photo-acoustic tomography of claim 1, wherein the light source is a semiconductor laser.

3. The photo-acoustic tomography of claim 1, wherein the amplification unit includes an optical-fiber optical amplifier.

4. A photo-acoustic tomography, comprising:
a first light source outputting first light to be absorbed in a biomaterial which is an inspection target;
a second light source outputting second light which has power equal to or lower than the power of the first light and has a lower frequency than the first light to be absorbed in the biomaterial;
a modulation unit controlling the power and the frequency of the second light output from the second light source;
a sensing unit sensing an ultrasonic wave generated as the first light and the second light are absorbed in the biomaterial; and
an image implementing unit implementing an image of an inner part of the biomaterial by using the ultrasonic wave sensed by the sensing unit.

5. The photo-acoustic tomography of claim 4, wherein the first light source and the second light source are semiconductor lasers.

6. The photo-acoustic tomography of claim 4, wherein the first light and the second light have a pulse type.

7. A photo-acoustic tomography, comprising:
a light source array including first to n-th light sources outputting first to n-th light to be absorbed in the biomaterial which is an inspection target;
a sensor array including first to m-th sensors sensing ultrasonic waves generated as the first to n-th light is absorbed in the biomaterial; and
an image implementing unit implementing an image of an inner part of the biomaterial by using the ultrasonic waves sensed by the sensor array (herein, n and m are integers of 2 or more).

8. The photo-acoustic tomography of claim 7, wherein the first to n-th light sources are semiconductor lasers.

9. The photo-acoustic tomography of claim 7, wherein the first to n-th light sources are disposed around the biomaterial in accordance with coordinates of (1,1), (1,2), . . . , (i,j).

10. The photo-acoustic tomography of claim 9, wherein the first to n-th light sources are disposed to configure a part of a sphere around the biomaterial.

11. The photo-acoustic tomography of claim 7, wherein the first to n-th light sources sequentially output light in accordance with a predetermined order or an arbitrary order.

12. The photo-acoustic tomography of claim 8, wherein the first to n-th light sources have different power.

13. The photo-acoustic tomography of claim 7, wherein the first to m-th sensors are disposed in spaces among the first to n-th light sources.

14. The photo-acoustic tomography of claim 8, wherein the first to m-th sensors are integrally configured with the first to n-th light sources.
